(19) United States
(12) Patent Application Publication
(10) Pub. No.: US 2019/0359547 A1
(43) Pub. Date: Nov. 28, 2019

(54) METHOD OF RETARDING AN ETHYLENE RESPONSE

(71) Applicant: CURTIN UNIVERSITY OF TECHNOLOGY, Bentley (AU)

(72) Inventors: Zora SINGH, Bentley (AU); Alan David PAYNE, Bentley (AU); Shamim Ahmed Kamal Uddin KHAN, Bentley (AU); Muftah Miload MUSA, Bentley (AU)

(73) Assignee: CURTIN UNIVERSITY OF TECHNOLOGY, Bentley (AU)

(21) Appl. No.: 16/333,757

(22) PCT Filed: Sep. 15, 2017
(86) PCT No.: PCT/AU2017/000197

§ 371 Date: Mar. 15, 2019

(30) Foreign Application Priority Data
Sep. 15, 2016 (AU) 2016903712

(51) Int. Cl. C07C 13/39; C07C 13/547; C07C 33/36; A01N 3/02; A01N 27/00

(52) U.S. Cl. CPC C07C 13/39; C07C 13/547; C07C 33/36; C07C 2603/08; A01N 27/00; C07C 2602/04; A01N 3/02

(57) ABSTRACT

A compound of Formula I, wherein R₁, R₂, R₃, R₄, R₅ and R₆ are independently hydrogen, halogen, an unsubstituted alkyl, alkenyl, alkynyl, cydoalkyi, cydoalkylalkyi, aryl, phenyl, or naphthyl group, and a substituted alkyl, alkenyl, alkynyl, cydoalkyi, cydoalkylalkyi, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

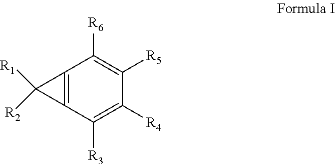

### TECHNICAL FIELD

A method of retarding an ethylene response in a plant.

### BACKGROUND ART

The following discussion of the background art is intended to facilitate an understanding of the present invention only. The discussion is not an acknowledgement or admission that any of the material referred to is or was part of the common general knowledge as at the priority date of the application.

Amongst different types of foods, fresh horticultural produce is highly perishable and postharvest losses (PHL) are up to 44% (0.57 billion tonnes) globally. Reduction of PHL will not only ensure food and nutritional security to the growing world population but also contributes to decrease the global warming through reduced use of land, water and other natural resources. The availability of high quality fresh fruits and vegetables at reasonable costs beyond the season could be ensured through the reduction of PHL from the farm gate to the consumers.

Ethylene promotes fruit ripening, senescence and abscission of plant organs and hence plays a key role in causing quantitative and qualitative postharvest losses in fresh horticultural produce. Usage of ethylene antagonists is one of the most effective approaches in retarding fruit ripening, extending postharvest life, maintaining quality and reducing PHL in fresh horticultural produce.

1-Methylcyclopropene (1-MCP) has been used commercially as an ethylene action inhibitor to retard fruit ripening and flower abscission. 1-MCP is also recommended for use in fresh horticultural produce. 1-MCP is a gas at room temperature and is highly unstable and difficult to use. In addition, it is not easily available to growers and is extremely expensive as its treatment is available only as a service not as a chemical.

### SUMMARY OF INVENTION

In accordance with the present invention, there is provided a compound of Formula I:

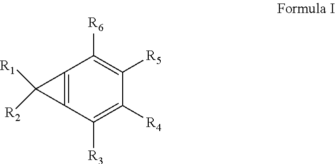

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In accordance with the present invention, there is provided a composition comprising a compound of Formula I:

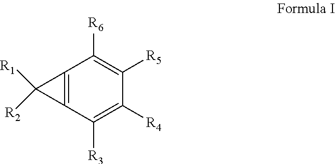

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In one form of the invention, at least one of R₁, R₂, R₃, R₄, R₅ and R₆ are independently alcohol. Preferably, at least one of R₁, R₂, R₃, R₄, R₅ and R₆ are independently polyols. Preferably, the at least one polyol is a sugar alcohol. Alternatively, the at least one polyol is a glycol such as ethylene glycol.

Preferably the alcohol is a polymerised alcohol such as polyethylene glycol.

In one form of the invention, R₁ and R₂ are independently an alkene or a ketone.

In one form of the invention, R₁ and R₂ are independently halogen.

In one form of the invention, R₁ and/or R₂ are hydrogen, fluorine and/or chlorine.

Preferably, R₃ and R₆ are hydrogen. Where R₃ and R₆ are hydrogen, R₄ and R₅ are preferably substituted.

In one form of the invention, the compound comprises at least one substituted or unsubstituted aromatic and/or nonaromatic ring formed between positions R₁ and R₂. Preferably, the ring is a carbocyclic or heterocyclic ring.

In one form of the invention, the compound comprises at least one substituted or unsubstituted aromatic and/or nonaromatic ring formed between positions R₃ and R₄, R₄ and R₅ and/or R₅ and R₆. Preferably, the ring is formed between positions R₄ and R₅.

In one form of the invention, R₁ and/or R₂ are hydrogen, fluorine and/or chlorine, R₃ and R₆ are hydrogen and R₄ and R₅ are substituted.

In one form of the invention, R₁ and/or R₂ are hydrogen, fluorine and/or chlorine, R₃ and R₆ are hydrogen and R₄ and R₅ form a substituted or unsubstituted aromatic and/nonaromatic ring.

In one form of the invention, R₁ and R₂ are fluorine and/or chlorine, and R₃, R₄, R₅ and R₆ are hydrogen.

In one form of the invention, there is provided a compound of Formula II:

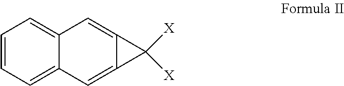

wherein X is hydrogen, fluorine and/or chlorine.

In one form of the invention, there is provided a compound of Formula III:

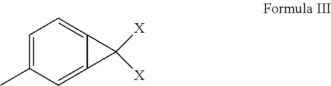

wherein X is hydrogen, fluorine and/or chlorine.

In one form of the invention, there is provided a compound of Formula IV:

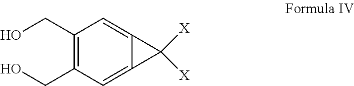

wherein X is hydrogen, fluorine and/or chlorine.

In one form of the invention, there is provided a compound of Formula V:

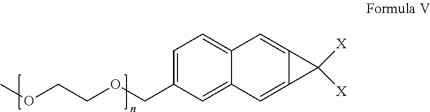

wherein X is hydrogen, fluorine and/or chlorine.

In one form of the invention, there is provided a compound of Formula VI:

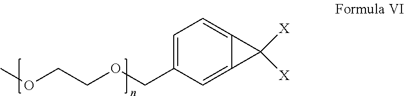

wherein X is hydrogen, fluorine and/or chlorine.

Preferably, the compound of the invention is water soluble.

In one form of the invention, the compound is provided in salt form.

Preferably, the salt of the compound is selected from the group comprising phosphate, acetate, formate, carbonate, hydrobromide, hydrochloride, sulfate, bisulfate, nitrate, acetate, trifluoroacetate, oxalate, valerate, oleate, palmitate, stearate, laurate, borate, benzoate, lactate, tosylate, citrate, maleate, fumarate, succinate, tartrate, naphthylate, mesylate, glucoheptonate, lactiobionate and laurylsulfonate salts.

In accordance with the present invention, there is provided a method for retarding an ethylene response in a plant comprising the step of contacting the plant with an effective ethylene response retarding amount of a compound of Formula I:

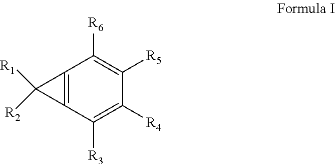

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In the context of the present specification, the term plant shall be understood to include whole plants and parts thereof, such as field crops, potted plants, cut flowers and fruits and vegetables as well as minimally processed fruits and vegetables.

The present invention can be employed to combat more than one different ethylene response. Ethylene responses may be initiated by either exogenous or endogenous sources of ethylene. Ethylene responses include, for example, the ripening and/or softening of fruits and vegetables, colour loss in vegetables, as well as minimally processed fruits and vegetables, shattering losses of pods and crop plants, senescence of flowers, abscission of foliage, flowers and fruit, the prolongation of the life of plants such as potted plants, cut flowers and dormant seedlings, the inhibition of growth and the stimulation of growth, adverse effects caused by stress [biotic and abiotic (wounding and mechanical stress, water stress, salinity, flooding/hypoxia, chilling, ozone injury)], degeneration of chlorophyll.

Ethylene responses or ethylene-type responses may also include increasing yields, increasing disease resistance, facilitating interactions with herbicides, increasing resistance to freeze injury, hormone or epinasty effects, hastening ripening and colour promotion in fruit, abscission of foliage, flowers and fruit, increasing flowering and fruiting, abortion or inhibition of flowering and seed development, prevention of lodging, stimulation of seed germination and breaking of dormancy, facilitating interactions with other growth regulators, auxin activity, inhibition of terminal growth, control of apical dominance, increase in branching, increase in tillering and changing biochemical compositions of plants.

Ethylene responses include, for example, the ripening and/or senescence of flowers, fruits and vegetables, abscission of foliage, flowers and fruit, the prolongation of the life of ornamentals such as potted plants, cut flowers, shrubbery, and dormant seedlings, the inhibition of growth and the stimulation of growth.

In accordance with the present invention, there is provided a method for retarding ripening of fruit comprising the step of contacting the fruit with an effective fruit ripening retarding amount of a compound of Formula I:

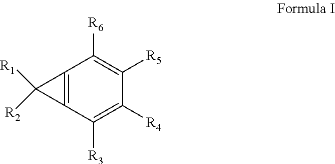

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In accordance with the present invention, there is provided a method for retarding ripening of vegetables comprising the step of contacting the vegetable with an effective vegetable ripening retarding amount of a compound of Formula I:

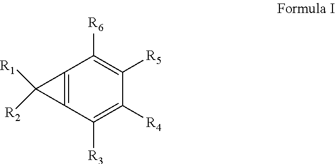

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In accordance with the present invention, there is provided a method for retarding senescence of a plant or plant part comprising the step of contacting the plant or plant part with an effective senescence retarding amount of a compound of Formula I:

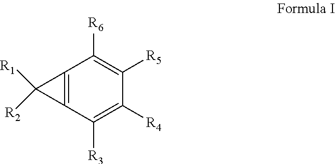

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In accordance with the present invention, there is provided a method for retarding abscission of a plant or plant part comprising the step of contacting the plant or plant part with an effective abscission retarding amount of a compound of Formula I:

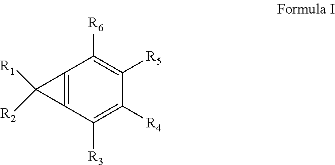

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In accordance with the present invention, there is provided a method for extending the life of a cut plant comprising the step of contacting the plant with an effective life extending amount of a compound of Formula I:

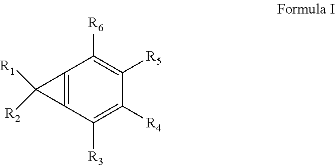

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

The method for extending the life of a cut plant comprising the step of contacting the plant with an effective life extending amount of Formula I may include extending the vase life of the cut plant.

In accordance with the present invention, there is provided a method for extending the storage life of fresh horticultural produce comprising the step of contacting the produce with an effective life extending amount of a compound of Formula I:

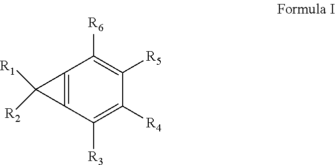

wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron.

In the context of the present invention, the term ripening shall be understood to encompass ripening of the fruit or vegetable while still on the relevant plant and the ripening after harvest.

The step of contacting the plant with an effective ethylene response retarding amount of Formula I may comprise dipping, spraying, irrigating or brushing at least a portion of the plant with or in a solution.

Fruits that may be treated by the method of the present invention include apple, nectarines, plums, tomatoes, apples, bananas, pears, papaya, mangoes, peaches, apricots, oranges, lemons, limes, grapefruit, tangerines, kiwifruit, pineapple, persimmon, avocados, melons, berries, cherries and other commercial cultivars, hybrids and new developed cultivars.

Vegetables that may be treated by the method of the present invention include leafy green vegetables such as lettuce, spinach and cabbage, roots such as potatoes and carrots, bulbs such as onions and garlic, herbs such as basil, oregano, dill, legumes such as soybean, lima beans and peas and corn, broccoli, cauliflower, asparagus and tomato.

Ornamental plants which may be treated by the method of the present invention to inhibit senescence and/or to prolong flower life and appearance (e.g., delay yellowing and abscission), include potted ornamentals, and cut flowers. Potted ornamentals and cut flowers which may be treated with the present invention include wax flowers, azalea, hydrangea, hybiscus, snapdragons, poinsettia, cactus, begonias, roses, tulips, daffodils, petunias, carnation, lily, gladiolus, alstroemeria, anemone, columbine, aralia, aster, bougainvillea, camellia, bellflower, cockscomb, falsecypress, chrysanthemum, clematis, cyclamen, freesia, and orchids of the family Orchidaceae and other commercial cultivars, hybrids and new developed cultivars.

Plants which may be treated by the method of the present invention include all temperate, topical and subtropical fruits, grapes and berry crops for example, apples, pears, mangos, cherries, pecans, grapes, olives, coffee, snapbeans, oranges, lemons, limes, grapefruit, tangerines and other commercial cultivars, hybrids and new developed cultivars, and weeping fig, as well as dormant seedlings such as various fruit trees including apple, ornamental plants, shrubbery, and tree seedlings.

In addition, shrubbery which may be treated according to the present invention to inhibit abscission of foliage include privet, photinea, holly, ferns, aglaonema, cotoneaster, barberry, waxmyrtle, abelia, acacia and bromeliades of the family Bromeliaceae, and other commercial cultivars, hybrids and new developed cultivars.

Fibre and oil seed crops which may be treated by the method of the present invention to inhibit abscission include cotton balls and seed shattering from pods in rapeseed, mustard and canola crops.

In accordance with the present invention, there is provided a composition for retarding an ethylene response in a plant comprising effective ethylene response retarding amount of a compound of Formula I in a substantially aqueous solution or an alcoholic solvent such as ethanol.

The composition may comprise one or more adjuvants such as carriers, extenders, binders, lubricants, surfactants, dispersants, wetting agents, spreading agents, dispersing agents, stickers, adhesives, defoamers, thickeners and emulsifying agents.

A carrier may be provided in the form of an organic solvent such as hydrocarbons and alcohols. Alternatively, the carrier may be in solid form such as talc or other inorganic, substantially inert materials, clays or zeolites.

Wetting agents may include various alkyl aryl sulfate salts, alkyl aryl sulfonate salts, polyalkyl alcohols.

The amount of the active ingredient required to inhibit the ethylene response will vary depending on numerous factors including the type of active ingredient, the type of ethylene response and the genotype and amount of plant material as well as the method of application. For fumigation purposes, a solution concentration would range from 0.01 nLL⁻¹ to 1000 μLL⁻¹ (v/v). More preferably, 1 nLL⁻¹ to 1000 nLL⁻¹. More preferably, 10 nLL⁻¹ to 100 nLL⁻¹. More preferably, 50 nLL⁻¹ to 100 nLL⁻¹. For spraying, dipping and waxing purposes, solution concentrations could range from 0.1 mgL⁻¹ to 1000 mgL⁻¹.

In one form of the invention, at least one of R₁, R₂, R₃, R₄, R₅ and R₆ are independently alcohol. Preferably, at least one of R₁, R₂, R₃, R₄, R₅ and R₆ are independently polyols. Preferably, the at least one polyol is a sugar alcohol. Alternatively, the at least one polyol is a glycol such as ethylene glycol.

Preferably the alcohol is a polymerised alcohol such as polyethylene glycol.

In one form of the invention, R₁ and R₂ are independently an alkene or a ketone.

In one form of the invention, R₁ and R₂ are independently halogen.

In one form of the invention, R₁ and/or R₂ are hydrogen, fluorine and/or chlorine.

Preferably, R₃ and R₆ are hydrogen. Where R₃ and R₆ are hydrogen, R₄ and R₅ are preferably substituted.

In one form of the invention, the compound comprises at least one substituted or unsubstituted aromatic and/nonaromatic ring formed between positions R₁ and R₂. Preferably, the ring is a carbocyclic or heterocyclic ring.

In one form of the invention, the compound comprises at least one substituted or unsubstituted aromatic and/nonaromatic ring formed between positions R₃ and R₄, R₄ and R₅ and/or R₅ and R₆. Preferably, the ring is formed between positions R₄ and R₅.

In one form of the invention, R₁ and/or R₂ are hydrogen, fluorine and/or chlorine, R₃ and R₆ are hydrogen and R₄ and R₅ are substituted.

In one form of the invention, R₁ and/or R₂ are hydrogen, fluorine and/or chlorine, R₃ and R₆ are hydrogen and R₄ and R₅ form a substituted or unsubstituted aromatic and/nonaromatic ring.

In one form of the invention, R₁ and R₂ are fluorine and/or chlorine, and R₃, R₄, R₅ and R₆ are hydrogen.

In one form of the invention, the compound is a compound of Formula II:

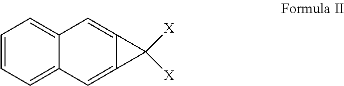

wherein X is hydrogen, fluorine and/or chlorine.

In one form of the invention, the compound is a compound of Formula III:

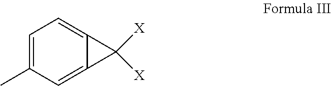

wherein X is hydrogen, fluorine and/or chlorine.

In one form of the invention, the compound is a compound of Formula IV:

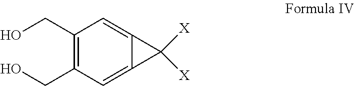

wherein X is hydrogen, fluorine and/or chlorine.

In one form of the invention, the compound is a compound of Formula V:

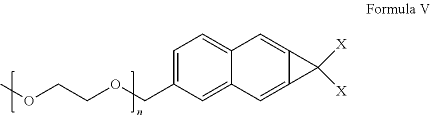

wherein X is hydrogen, fluorine and/or chlorine.

In one form of the invention, the compound is a compound of Formula VI:

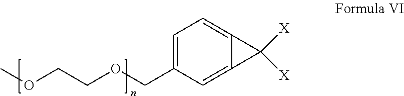

wherein X is hydrogen, fluorine and/or chlorine.

Preferably, the compound of the invention is water soluble.

In one form of the invention, the compound is provided in salt form.

Preferably, the salt of the compound is selected from the group comprising phosphate, acetate, formate, carbonate, hydrobromide, hydrochloride, sulfate, bisulfate, nitrate, acetate, trifluoroacetate, oxalate, valerate, oleate, palmitate, stearate, laurate, borate, benzoate, lactate, tosylate, citrate, maleate, fumarate, succinate, tartrate, naphthylate, mesylate, glucoheptonate, lactiobionate and laurylsulfonate salts.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is the chemical structures of benzocyclopropene and naphtho[b]cyclopropane;

FIG. 2 depicts the concentration of ethylene and CO₂ on the day of climacteric peak;

FIG. 3 depicts the concentration of ethylene (A) and CO₂ (B) on the day of climacteric peak in ‘Fortune’ plum fruit treated with different concentration of naphtho[b]cyclopropane (NC); and

FIG. 4 depicts the percent of flower/bud abscission after two days of treatment with antagonist.

### DESCRIPTION OF EMBODIMENTS

Throughout this specification, unless the context requires otherwise, the word “comprise” or variations such as “comprises” or “comprising”, will be understood to imply the inclusion of a stated integer or group of integers but not the exclusion of any other integer or group of integers.

Benzocyclopropene was prepared as shown in Scheme 1.

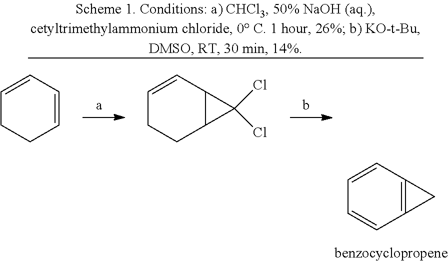 [SMILES: C1=CCCC=C1 ; ClC1(Cl)C2C=CCCC21 ; c1ccc2c(c1)C2]

A solution of 1,3-cyclohexadiene (80.5 g, 0.5 mol), cetyltrimethylammonium bromide (2.00 g) in 50% aqueous sodium hydroxide (200 g) was cooled (0-25° C.) and stirred under nitrogen. Ethanol (5 mL) and chloroform (80 mL) were added successively in one portion. The solution was stirred for 1 hour at 0° C. and allowed to warm to room temperature for a further 1 hour. Water was added to the reaction mixture and extracted. The organic phase was washed with water (2×100 mL), dried (CaCl₂) and concentrated under reduced pressure to provide an oil. The oil was purified by flash chromatography to afford the 7,7-dichlorobicyclo[4.1.0]-hept-2-ene as a colourless oil (42 g, 26%).

Potassium t-butoxide was added in portions to a solution of 7,7-dichlorobicyclo[4.1.0]-hept-2-ene (1.00 g, 6.1 mmol) in anhydrous DMSO (30 mL) under nitrogen. The dark brown mixture was stirred for 30 minutes. A vacuum was applied to the reaction mixture and the volatiles collected in an −86° C. trap. The distillate was diluted in petroleum spirits, and washed with brine (4×60 mL) and water (2×30 mL), dried (Na₂SO₄) and concentrated under reduced pressure at 0° C. to afford benzocyclopropene as an oil (80 mg, 14%). 1H NMR (CDCl₃) δ 3.17 (2H, s), 7.21 (s, 4H).

Naphtho[b]cyclopropene was prepared as shown in Scheme 2.¹ ¹W. E. Billups and C. Y. Chow J. Am. Chem. Soc. 1973, 95, 4099.

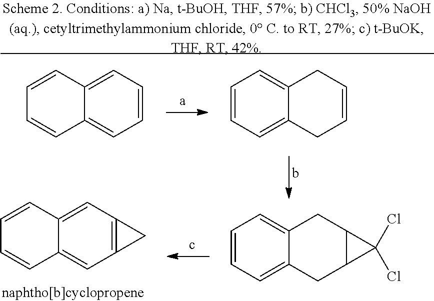 [SMILES: c1ccc2ccccc2c1 ; C1=CCc2ccccc2C1 ; c1ccc2cc3c(cc2c1)C3 ; ClC1(Cl)C2Cc3ccccc3CC21]

Small pieces of sodium metal (15 g) were added to solution of naphthalene (30 g) in anhydrous THF (100 mL). The solution turned to a deep green colour during this time. A solution of tert-butanol (24 mL) and THF (24 mL) in water was added dropwise over 20 minutes. The resulting solution was stirred for a further 3 hours. The excess sodium metal was removed by filtration and the filtrate washed with water (2×50 mL), dried and concentrated under reduced pressure to give pure 1,4-dihydronaphthalene as a colourless solid (17.5 g, 57%).

A solution of 1,4-dihydronaphthalene (17.0 g, 0.131 mol), cetyltrimethylammonium bromide (0.567 g, 1.6 mmol) in 50% aqueous sodium hydroxide (50 g) was cooled (0-25° C.) and stirred under nitrogen. Ethanol (1.6 mL) followed by chloroform (23 mL) was added. The solution was stirred for 1 hour at 0° C. and allowed to warm to room temperature for a further 1 hour. Water (100 mL) was added to the reaction mixture and extracted. The organic phase was washed with water (2×50 mL), dried (CaCl₂) and concentrated under reduced pressure to provide an oil. The oil was purified by flash chromatography to afford the adduct (7.56 g, 27%).

Potassium tert-butoxide (11.0 g, 98.2 mmol) was added in portions to a solution of 1,1-dichloro-1a,2,7,7a-tetrahydro1H-cyclopropa[b]naphthalene (4.78 g, 29.3 mmol) in anhydrous THF (60 mL) under nitrogen at room temperature and stirred for a further 18 hour. The resulting mixture was diluted in petroleum spirits (20 mL), washed with brine (4×20 mL) and water (2×10 mL), dried (Na₂SO₄) and concentrated under reduced pressure. The residue was purified by flash chromatography to afford naphtho[b]cyclopropene as a colourless solid (1.11 g, 42%).

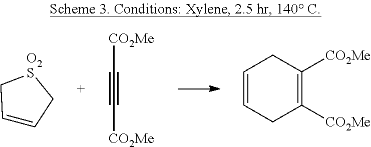 [SMILES: O=S1(=O)CC=CC1 ; COC(=O)C#CC(=O)OC ; COC(=O)C1=C(C(=O)OC)CC=CC1]

A solution of dimethyl acetylenedicarboxylate (1.00 mL, 0.008 mol), butadiene sulfone (5.40 g, 0.045 mol) and xylene (8 mL) was heated under reflux under nitrogen atmosphere for 2.5 hr. After 2.5 hr, the yellow reaction mixture was allowed to cool to room temperature and concentrated under reduced pressure. The residue was purified by column chromatography (10% ethyl acetate/petroleum spirits) to give dimethyl-1,4-cyclohexadiene-1,2-dicarboxylate as a yellow liquid (0.75 g, 47%). ¹H NMR: δ 5.93 (m, 1H), 3.78 (s, 3H), 3.00 (d, J=1.2 Hz, 4H).

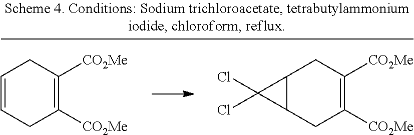 [SMILES: COC(=O)C1=C(C(=O)OC)CC=CC1 ; COC(=O)C1=C(C(=O)OC)CC2C(C1)C2(Cl)Cl]

1,4-Cyclohexadiene-1,2-dicarboxylate (0.20 g, 0.1 mmol) was added to a solution of sodium trichloroacetate (3 g, 16 mmol) and tetrabutylammonium iodide (0.005 g, 0.014 mmol) in chloroform (20 mL) and the reaction mixture was heated under reflux under nitrogen overnight. The reaction was allowed to cool to room temperature and diluted in chloroform (20 mL), washed with water (5×25 mL), dried under anhydrous calcium chloride and the solvent was removed under reduced pressure. The residue was purified by column chromatography (10% ethyl acetate/petroleum spirits) to afford dichlorocarbene adduct as a colourless solid (0.172 g, 87%). ¹H NMR: δ 3.76 (s, 6H); 2.87-2.75 (m, 2H); 2.55 (d, J=7.6 Hz, 2H); 1.98-1.94 (m, 2H). ¹³C NMR δ: 168.11 (C); 131.68 (C); 64.06 (C); 52.37 (CH); 23.84 (CH₂); 21.56 (CH₃). IR: 3058, 2873, 1736, 1628, 1600, 1469, 1390, 1352, 1258, 1216, 1104, 970, 838, 747. Microanalysis: Calculated: C=47.33, H=4.33%, Found: C=47.32, H=4.11%.

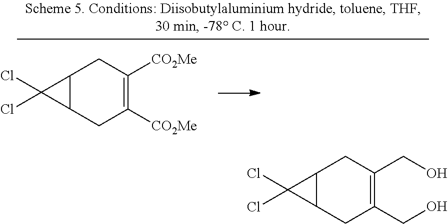 [SMILES: COC(=O)C1=C(C(=O)OC)CC2C(C1)C2(Cl)Cl ; OCC1=C(CO)CC2C(C1)C2(Cl)Cl]

Diisobutylaluminium hydride (1.364 g, 0.01 mol) and toluene solution was added dropwise to a solution of the diester (0.10 g, 0.0004 mol) in dry THF (10 mL) at −78° C. The reaction was stirred for 30 minutes at −78° C., and then it was allowed to warm to room temperature overnight. 1 M HCl solution (10 mL) was added to the reaction mixture and extracted with ethyl acetate (3×20 mL). The combined organic extracts were dried over anhydrous magnesium sulfate and concentrated under reduced pressure. The residue was purified by column chromatography (40% ethyl acetate in petroleum spirit) to give 7,7-Dichloro-bicyclo[4,1,0]hept-3-ene-1,2-dimethanol as colourless solid (0.038 g, 46%). ¹H NMR: δ 4.23 (m, 4H); 2.74-2.58 (m, 2H); 2.34 (d, J=10.9 Hz, 2H); 1.89 (d, J=10.9 Hz, 2H). ¹³C NMR δ: 13104 (C), 65.31 (C), 62.55 (CH), 24.83 (CH), 23.53 (CH₂). IR: 3291, 2878, 2830, 1679, 1422, 1261, 1086, 995, 784. Microanalysis: Calculated: C=48.45, H=5.42%; Found: C=48.32, H=5.33%.

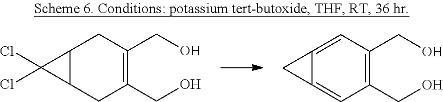 [SMILES: OCC1=C(CO)CC2C(C1)C2(Cl)Cl ; OCc1cc2c(cc1CO)C2]

7,7-Dichloro-bicyclo[4,1,0]hept-3-ene-1,2-dimethanol (0.0485 g, 0.2 mmol) was added to a solution of potassium tert-butoxide (0.154 g, 1.4 mmol) in dry THF (8 mL) the reaction mixture was stirred for 36 hrs. Petroleum spirits (10 mL) was added to the brown mixture and washed with brine water (3×10 mL) and water (1×10 mL) and then the organic phase was dried under anhydrous magnesium sulfate and the solvent was removed under reduced pressure to give 1H-cyclopropabenzene-3,4-dimethanol. ¹H NMR: δ 7.27 (s, 2H); 4.79 (s, 4H); 3.28 (s, 4H).

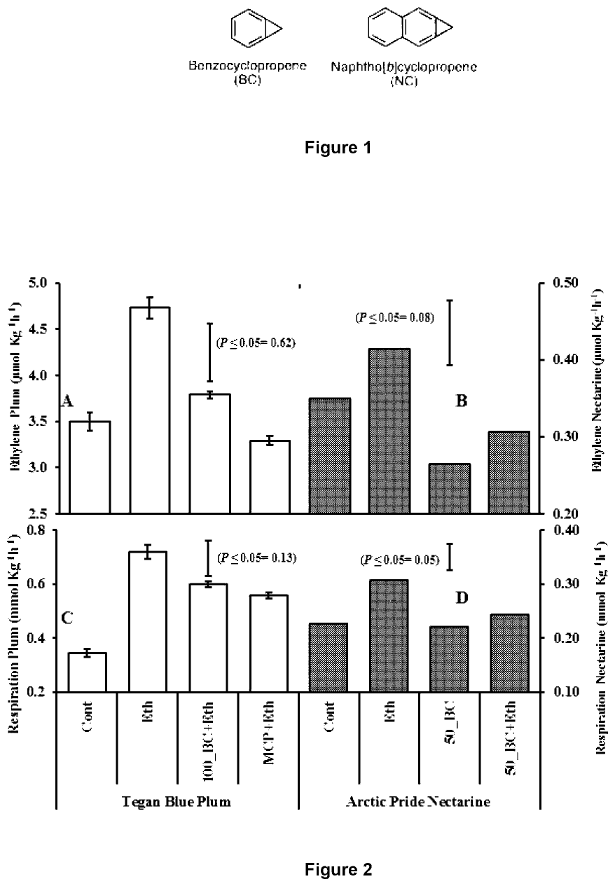

The chemical structures of benzocyclopropene (BC) and naphtho[b]cyclopropene (NC) are provided at FIG. 1. Advantageously, benzocyclopropene is a liquid at room temperature, making it easier to handle than 1-methylcyclopropene. Advantageously, naphthocyclopropene is a solid at room temperature, making it easier to handle than 1-methylcyclopropene.

Advantageously, benzocyclopropene and naphtho[b]cyclopropane are stable at room temperature for several months.

Benzocyclopropene and naphtho[b]cyclopropene are only partially soluble in water. To prepare substantially aqueous solutions of these compounds, the lead compounds where dissolved in ethanol and then diluted with water.

Various experiments were conducted using ‘Tegan Blue’ and ‘Fortune’ Japanese plums, ‘Arctic Pride’ nectarine, Fuji and Pink Lady™ apples, WX7, WX17, WX39, WX 56, WX 58, WX73 and WX107, WXFU, hybrid, Revelation, Purple Pride and Jenny wax flowers (Chamelaucium Desf.).

Mature ‘Tegan Blue’ and ‘Fortune’ Japanese plum and ‘Arctic Pride’ nectarine fruits of uniform size and maturity, free from visual blemishes and diseases were harvested in early morning from a commercial orchard in Western Australia. Following the harvest, the fruit were brought to the Horticulture Research Laboratory, Curtin University, using a temperature controlled vehicle at 20-25° C. The fruit were treated by fumigation with 0 to 100 nLL⁻¹ of BC and 1000 nLL⁻¹ of 1-MCP (Tegan Blue plum) for 18 hr at ambient conditions (20±1° C. and 65±5% RH) by using hermetically sealed plastic containers of 60 L volume. Whatman filter paper (number 2) soaked with specific concentrations of BC and 1-MCP were kept along with the fruit and 30 g of soda lime inside each container. A small battery operated fan was used to ensure equal distribution of the vapours from the chemicals. Half of the treated fruit was exposed to ethylene (10 μL L⁻¹) for 24 hr following BC and 1-MCP treatment.

In a second set of experiments, ‘Fortune’ plum fruit were treated with 0 to 1000 nIL⁻¹ of NC as described above. The treated fruit were kept in the ambient conditions for ripening and endogenous level of ethylene and CO₂ was determined. The experiments were laid out by following completely randomized design (CRD) with four replications for each treatment and 10 fruit in each replication.

To evaluate the effects of BC and NC on flower abscission, flowering stems of Wax flower (Chamelaucium Desf.) (WX17, WX73 and WX107) were collected from mature bushes grown at Department of Agriculture and Food Western Australia (DAFWA), Perth (31° 58′ 55″ S/115° 51′ 47″ E). Collected stems were immediately placed upright in buckets with water and recut at 20-25 cm in length (from the cut end to the most extreme opened-flowers). The flower stalks were treated similarly as the fruits with BC (0-100 nLL⁻¹) or NC (0-100 nLL⁻¹) and ethylene. The experiments were laid out by following CRD design, having five replications for each treatment and five stalks in each replication. During the treatment period, the flower stalks were placed in small plastic bottles with distilled water. A cone made of nylon mesh was placed at the base of the stalks to check the number of abscised flowers.

The endogenous level of ethylene was determined by using the Sensor Sense (Sensor sense B.V, Nijmegen, The Netherlands). The Sensor Sense includes an ETD 300 ethylene detector, a set of valve controllers with an option of six valves connected to six separate cuvettes [1.0 L air-tight jar, fitted with a rubber septum (SubaSeal®, Sigma-Aldrich Co., St. Louis, USA)]. The continuous flow method was used with coarse mode (conversion factor 99818, capacity to measure ethylene concentration at 0-500 ppm, sensitivity at <1%) of analysis. Each sample was run for 20 minutes with a flow rate of 4.0 L hour⁻¹ and the average reading of last 15 minutes was considered to calculate the concentration of ethylene and expressed as μmol kg⁻¹ h⁻¹.

Respiration rate was determined as carbon dioxide (CO₂) production from the fruit during ripening period a using CO₂ analyser. The headspace gas sample (2.0 mL) was taken through rubber septum (SubaSeal®, Sigma-Aldrich Co., St. Louis, USA) using a syringe from the air tight jar with sample fruit and injected into an infrared gas analyser [Servomex Gas Analyzer, Analyzer series 1450 Food Package Analyzer, Servomex (UK) Ltd., East Sussex, UK]. The respiration rate was calculated on the basis of the peak areas of 2.0 mL gas sample and CO₂ standard (8.52±0.17%) and expressed as mmoL CO₂ kg⁻¹ h⁻¹.

Assessment of floral organs abscission (%): Following ethylene treatment (2-4 days), the flower stalks were taken out from the treatment container and gently beaten against a collection tray to calculate the percentage of abscised flowers and buds.

The experimental data were analysed following one-way analysis of variance (ANOVA) by using Genstat 13 (release 13.1; Lawes Agricultural Trust, Rothamsted Experimental Station, Harpenden, UK). The effects of various treatments and their interactions were assessed and least significant differences (Fisher's LSD) were calculated by F test at 5% level of significance.

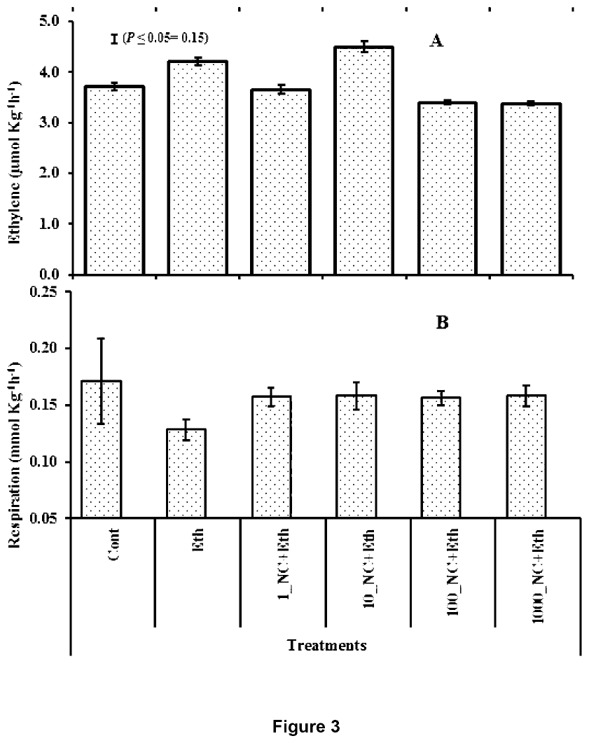

The level of climacteric ethylene in ‘Tegan Blue’ plum fruit was significantly (P 0.05) suppressed by 100 nLL⁻¹ BC+ethylene and 1000 nLL⁻¹ 1-MCP+ethylene (0.80- and 0.70-fold respectively) in comparison to the solely ethylene treated fruit where the ethylene concentration was 4.73 μmol kg⁻¹ h⁻¹ (FIG. 2A).

Similarly, BC (50 nLL⁻¹)+ethylene treated ‘Arctic Pride’ nectarine fruit exhibited significantly suppressed (0.63-fold) levels of ethylene than the solely ethylene treated fruit (0.414 μmol kg⁻¹ h⁻¹) (FIG. 2 B).

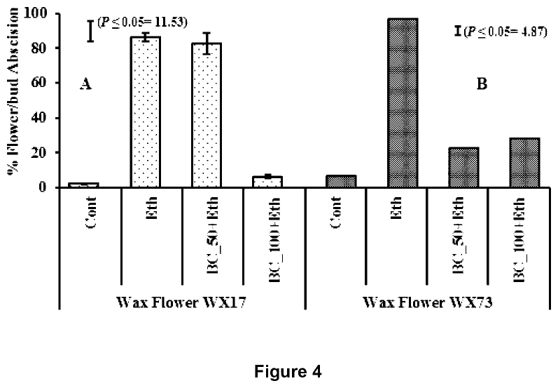

The NC (100-1000 nLL-1) also showed antagonistic effect by significantly suppressing the level of climacteric ethylene (0.81-fold) than the solely ethylene treated fruit in ‘Fortune’ plum fruit (FIG. 3).

The climacteric respiration was also suppressed in BC (100 nLL⁻¹)+ethylene and 1-MCP (1000 μLL⁻¹)+ethylene treated ‘Tegan Blue’ plum fruit (0.83- and 0.77-fold respectively) than the solely ethylene treated fruit (0.72 mmol CO₂ kg⁻¹ h⁻¹) (FIG. 2C). On the other hand, significant suppression of respiration climacteric was observed in both BC (50 nLL⁻¹) and BC (50 nLL⁻¹)+ethylene treated ‘Arctic Pride’ nectarine fruit (0.71- and 0.77-fold respectively) in comparison to the solely ethylene treated fruit (0.31 mmol CO₂ kg⁻¹ h⁻¹) (FIG. 2D).

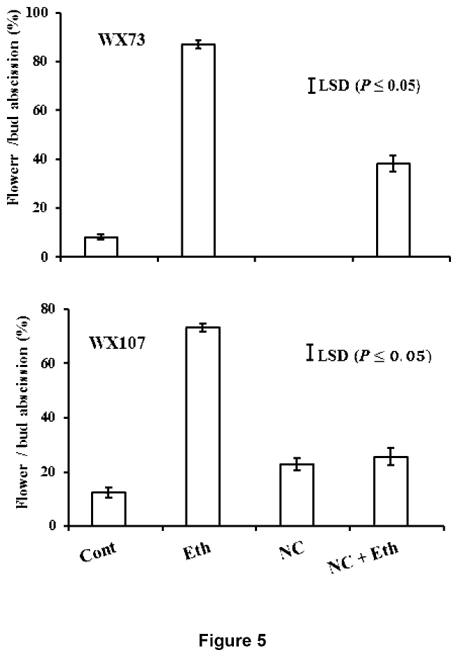

The fumigation of BC (100 nLL⁻¹) followed by ethylene exposure (10 μLL⁻¹) significantly reduced the rate of flower/bud abscission in WX17 (6.05%). Whilst 50 and 100 nL L⁻¹ BC followed by ethylene treatment significantly lowered the rate of abscission at 22.43% and 28.40% respectively in WX73 wax flower as compared to ethylene treatment alone (FIGS. 4 A and B).

The treatment of NC (100 nL L⁻¹) also significantly (P≤0.05) suppressed the rate of flower/bud abscission in WX73 (0%) and WX107 (22.82%) wax flowers in comparison to the ethylene treated flowers. Suppressed flower/bud abscission was also observed in WX73 and WX107 wax flowers (38.11% and 25.51% respectively), even when the NC treatment was followed by ethylene treatment (10 μL L⁻¹) (FIG. 5). The highest level of flower/bud abscission in all genotypes was noted from the ethylene treated flowers.

Fruit and flower stalks treated with BC or NC (50-100 nL L⁻¹) followed by ethylene treatment (10 μL L⁻¹) significantly (P≤0.05) reduced the rate of flower abscission and concentration of climacteric ethylene and CO₂ production than the solely ethylene exposed flowers and fruits which suggests that the inhibition of ethylene action by the BC and NC was not only exogenous but also at endogenous. This is the first disclosure on the effects of BC and NC on antagonising ethylene action during fruit ripening and floral organs abscission processes.

Similar effects to 1-MCP have been observed for BC and NC which has been reflected through the non-significant differences among the effects of BC (100 nL L⁻¹) and 1-MCP (1000 nL L⁻¹) on climacteric ethylene in ‘Tegan Blue’ and ‘Fortune’ Japanese plum fruits (FIGS. 2 A and 3 A) ‘Arctic Pride’ nectarine (FIG. 2 B) and respiration in ‘Tegan Blue’ plum (FIG. 2C) and ‘Arctic Pride’ nectarine (FIG. 2 D).

BC and NC fumigation exhibited ethylene antagonistic effects on ripening of climacteric fruits such as plums, nectarines and abscission of floral organs in wax flowers.

In the tested chemicals (BC and NC), the cyclopropene portion of the molecule is thought to make a potential bond at or near the ethylene binding site of the receptor. As the flowers/fruit were exposed to the BC and NC treatment shortly after collection and completely blocked the ethylene receptor to prohibit ethylene activity for a period of time, so it worked at a non-competitive basis.

1. A compound of Formula I: 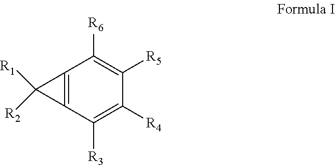
wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron,
or a salt thereof.

2. A composition comprising a compound of Formula I: 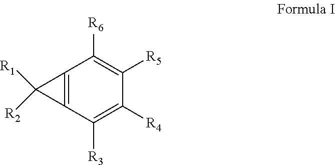
wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron,
or a salt thereof.

3. The compound or salt in accordance with claim 1, wherein at least one of R₁, R₂, R₃, R₄, R₅ and R₆ are independently alcohol.

4. The compound or salt in accordance with claim 1, wherein at least one of R₁, R₂, R₃, R₄, R₅ and R₆ are independently polyols.

5. The compound or salt in accordance with claim 4, wherein each polyol is independently selected from the group consisting of a sugar alcohol and a glycol such as ethylene glycol.

6. (canceled)

7. The compound or salt in accordance with claim 3, wherein the alcohol is a polymerised alcohol such as polyethylene glycol.

8. The compound or salt in accordance with claim 1, wherein R₁ and R₂ are independently selected from an alkene, a ketone, a halogen and hydrogen.

9. (canceled)

10. (canceled)

11. The compound or salt in accordance with claim 1, wherein R₃ and R₆ are hydrogen.

12. The compound or salt in accordance with claim 11, wherein R₄ and R₅ are substituted.

13. The compound or salt in accordance with claim 1, wherein the compound comprises at least one substituted or unsubstituted aromatic and/or nonaromatic ring formed between positions R₁ and R₂.

14. The compound or salt in accordance with claim 13, wherein the ring is a carbocyclic or heterocyclic ring.

15. The compound or salt in accordance with claim 1, wherein the compound comprises at least one substituted or unsubstituted aromatic and/or nonaromatic ring formed between positions R₃ and R₄, R₄ and R₅ and/or R₅ and R₆.

16. (canceled)

17. (canceled)

18. (canceled)

19. (canceled)

20. The compound or salt in accordance with claim 1, wherein there is provided a compound of Formula II: 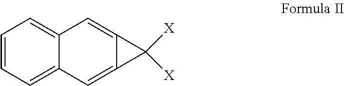
wherein X is hydrogen, fluorine and/or chlorine.

21. The compound or salt in accordance with claim 1, wherein there is provided a compound of Formula III: 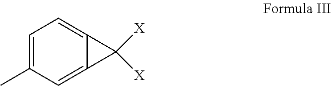
wherein X is hydrogen, fluorine and/or chlorine.

22. The compound or salt in accordance with claim 1, wherein there is provided a compound of Formula IV: 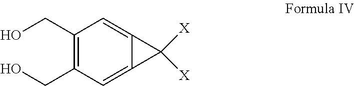
wherein X is hydrogen, fluorine and/or chlorine.

23. The compound or salt in accordance with claim 1, wherein there is provided a compound of Formula V: 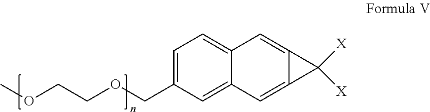
wherein X is hydrogen, fluorine and/or chlorine.

24. The compound or salt in accordance with claim 1, wherein there is provided a compound of Formula VI: 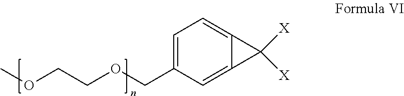
wherein X is hydrogen, fluorine and/or chlorine.

25. The compound or salt in accordance with claim 1, wherein the compound of the invention is water soluble.

26. (canceled)

27. The compound or salt in accordance with claim 261, which is provided in the form of a salt selected from the group comprising phosphate, acetate, formate, carbonate, hydrobromide, hydrochloride, sulfate, bisulfate, nitrate, acetate, trifluoroacetate, oxalate, valerate, oleate, palmitate, stearate, laurate, borate, benzoate, lactate, tosylate, citrate, maleate, fumarate, succinate, tartrate, naphthylate, mesylate, glucoheptonate, lactiobionate and laurylsulfonate salts.

28. A method for one or more of:
retarding an ethylene response in a plant;
retarding ripening of fruit;
retarding ripening of vegetables;
retarding senescence of a plant or plant part;
retarding abscission of a plant or plant part;
extending the life of a cut plant; and
extending the storage life of fresh horticultural produce;
the method comprising the step of contacting the plant with an effective ethylene response retarding amount of a compound of Formula I: 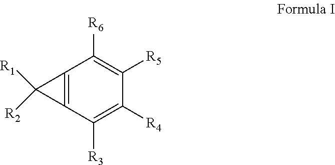
wherein R₁, R₂, R₃, R₄, R₅ and R₆ are independently hydrogen, halogen, an unsubstituted alkyl, alkenyl, alkynyl, cycloalkyl, cycloalkylalkyl, aryl, phenyl, or naphthyl group, and a substituted alkyl, alkenyl, alkynyl, cycloalkyl, cycloalkylalkyl, aryl, phenyl, or naphthyl group having as a substituent a halogen, alkoxy, substituted phenoxy, unsubstituted phenoxy group or a heteroatom such as oxygen, sulfur, nitrogen, phosphorus and boron,
or a salt thereof.

29. (canceled)

30. (canceled)

31. (canceled)

32. (canceled)

33. (canceled)

34. (canceled)
